(19) United States
(12) Patent Application Publication
(10) Pub. No.: US 2015/0209274 A1
(43) Pub. Date: Jul. 30, 2015

(54) DRUG ELUTING MEMBER, A METHOD OF ATTACHING THE SAME AND A METHOD OF FABRICATING THE SAME, A DEVICE FOR HOLDING THE SAME AND A DRUG ELUTING DEVICE

(71) Applicants: Nanyang Technological University, Singapore (SG); Singapore Health Services Pte Ltd, Singapore (SG)

(72) Inventors: Subramanian Venkatraman, Singapore (SG); Yan Peng, Singapore (SG); Tina Tzee Ling Howden, Singapore (SG)

(21) Appl. No.: 14/421,387

(22) PCT Filed: Aug. 14, 2013
(86) PCT No.: PCT/SG2013/000346

§ 371 Date: Feb. 12, 2015

Related U.S. Application Data
(60) Provisional application No. 61/683,438, filed on Aug. 15, 2012.

(51) Int. Cl. A61K 9/00; A61K 31/4709; B32B 37/22; A61K 47/34; B25J 1/04; A61K 31/5383; A61K 31/496

(52) U.S. Cl. CPC A61K 9/0051; A61K 31/5383; A61K 31/4709; A61K 31/496; A61K 47/34; B25J 1/04; B32B 37/22; B32B 2264/00

(57) ABSTRACT

A drug eluting member is adapted to be attachable onto a perimeter edge of a lens portion of an intraocular lens, the drug eluting member including an interfacing portion adapted to receive a portion of the perimeter edge. A drug eluting device includes a first and second drug eluting members adapted to be attached to first and second portions, respectively of the perimeter edge. A method attaches the drug eluting member to the perimeter edge by positioning a holding device with the drug eluting member held therein against a portion of the perimeter edge; and releasing the drug eluting member from the holding device portion. A method of fabricating the drug eluting member includes providing a mold for molding the drug eluting member; discharging a forming solution from a nozzle onto the mold; and forming the drug eluting member.

### CROSS-REFERENCE TO RELATED APPLICATIONS

The present application claims the benefit of the U.S. provisional patent application No. 61/683,438 filed on 15 Aug. 2012, the entire contents of which are incorporated herein by reference for all purposes.

### TECHNICAL FIELD

The present invention relates to a drug eluting member, a method of attaching the drug eluting member and a method of fabricating the drug eluting member. In addition, the present invention provides a device for holding the drug eluting member and a drug eluting device.

### BACKGROUND

Cataract may be the first and a major cause for reversible blindness and the cataract-affected population has been on the rise globally due to increasing life expectancy. Intraocular lens (IOL) market accounted for the largest share, which was 77.7%, of the overall cataract devices market at $3.4 billion in year 2009, and this number is expected to reach $5.2 billion in year 2014.

IOL can restore the patients' vision, nevertheless, post-operative infection, such as bacterial endophthalimitis may result in devastating permanent vision loss. A typical current solution to manage the post-operative infection is to administer a short course of antibiotics topically or deliver the antibiotics intracamerally at the end of the operation. However, due to the low level of intraocular penetration (less than 0.3%) from topical application, a very high concentration of antibiotics needs to be applied, which is costly and toxic to the ocular tissues. Moreover, patient non-compliance with frequent administration of the eye drops becomes a major issue that potentially leads to suboptimal therapeutic effect.

Only in recent years, researchers started to recognise this problem and focus on the potential development of a drug loaded IOL to overcome the problems generated by the use of eye drops. One attempt in achieving a drug loaded IOL was to soak the commercial IOLs in gatifloxacin and levofloxacin solutions, which only achieved therapeutic concentration in 72 hours. Other attempts were to adopt the soaking method, but none of them were able to release the active agents for a period longer than a week. In addition to a limited period of drug release, the soaking method may also generate an undesirable huge initial burst release of the antibiotics which may be toxic to the surrounding ocular tissues. Another approach was developed utilising a biodegradable drug loaded tube on the haptic/s of the IOL. Despite of the longer period of release, the orientation and centration of the lens may be distorted upon the IOL insertion due to unbalanced weight. Therefore, none of these approaches are able to achieve a sustained release over a period of two weeks, without potential affecting the optical properties and distorting the orientation of the implanted IOL.

Topical administration of antibiotics post-cataract surgery needs to be frequent. Generally, antibiotic eye drop is applied 4-6 times daily over two weeks after the natural crystal lens has been replaced by synthetic IOL due to cataract. Preservative in the eye drop and non-compliance by patients may lead to serious problems. Current research on the drug eluting intraocular lens does not seem to achieve a sustained release over the required minimum administration period, e.g. fourteen days, without affecting the optical properties of the IOLs or the configuration of the IOLs post-implantation.

### SUMMARY

The present invention provides a drug eluting member adapted to be attachable onto a perimeter edge of a lens portion of an intraocular lens, the drug eluting member includes an interfacing portion adapted to receive a portion of the perimeter edge.

According to various embodiments, the interfacing portion includes a channel that is adapted to receive the portion of perimeter edge therein.

According to various embodiments, the channel conforms to the profile of the portion of the perimeter edge.

According to various embodiments, the interfacing portion includes an adhesive surface adapted to adhere the drug eluting member to the lens portion.

According to various embodiments, the drug eluting member is bio-degradable.

According to various embodiments, the drug eluting member is arcuated.

According to various embodiments, the drug eluting member surrounds the lens portion along the perimeter edge of the lens portion.

According to various embodiments, the drug eluting member is ring-shaped.

The present invention provides a drug eluting device that includes a first drug eluting member and a second drug eluting member of any one of drug eluting members referred to above such that the first drug eluting member is being adapted to be attached to a portion of a perimeter edge of a lens portion of an intraocular lens, and the second drug eluting member is being adapted to be attached to another portion of the perimeter edge.

According to various embodiments, when attached to the lens portion, the first drug eluting member is substantially opposite the second drug eluting member.

According to various embodiments, the first drug eluting member and second drug eluting member meet to form a through hole capable of surrounding the lens portion of the intraocular lens thereby attaching the first eluting member and second eluting member to the lens portion.

The present invention provides a device for holding any one of the drug eluting members referred to above to a portion of a perimeter edge of a lens portion of an intraocular lens, the device includes a holder being adapted to hold the drug eluting member; and a handling portion for a user to handle the holder.

According to various embodiments, the holder includes a receiving channel for receiving the drug eluting member therein.

According to various embodiments, the handling portion includes a handle extending from the holder.

The present invention provides a method of attaching any one of the drug eluting members referred to above to a perimeter edge of a lens portion of an intraocular lens using any one of the devices referred to above, the method includes positioning the device with the drug eluting member held therein against a portion of the perimeter edge; and releasing the drug eluting member from the device to attach the drug eluting member to the lens portion.

According to various embodiments, the method further includes the step of adhering the drug eluting member to the lens portion.

The present invention provides a method of fabricating any one of the drug eluting member referred to above, the method includes providing a mold for molding the drug eluting member; discharging a forming solution from a nozzle onto the mold; and forming the drug eluting member.

According to various embodiments, the step of forming the drug eluting member includes displacing the mold with respect to the nozzle; and coating the mold with the forming solution.

According to various embodiments, the step of displacing the mold includes rotating the mold at about an axis; and/or translating the mold along the axis.

According to various embodiments, the method further includes the step of shaping the drug eluting member.

According to various embodiments, the mold is a disc-shaped plate.

According to various embodiments, the mold is a disc-shaped plate with an augmented perimeter portion such that the thickness of the mold at the augmented perimeter portion is larger than the thickness of the mold at the centre portion.

According to various embodiments, the forming solution includes a polymer and drug solution.

The drug eluting member of the present invention may be attached or adhered to the existing commercially available IOLs; and may be completely biodegradable with drug elution locally over the required administration period. Unlike other approaches, the drug eluting member of the present invention does not seem to affect the optical properties of the IOL, nor change the mass of the entire IOL (including the haptics). The design may be applicable to any intra-ocular lens design, regardless of the intraocular lens material, degree of the intraocular lens, dimension of the intraocular lens.

### BRIEF DESCRIPTION OF DRAWINGS

FIG. 1 shows an elevation view exemplary embodiment of a drug eluting member attached to an intraocular lens;

FIG. 2 shows a sectional view of the drug eluting member in FIG. 1;

FIG. 3 shows a frontal view of the drug eluting member in FIG. 1;

FIG. 4a-4c show various view of an exemplary embodiment of a drug eluting member;

FIG. 5 shows a perspective view of the drug eluting members in FIG. 4a attached to an intraocular lens;

FIG. 6 shows an elevation view of an exemplary embodiment of a drug eluting member;

FIG. 7a-7b show various views of the drug eluting member in FIG. 6 attached to an intraocular lens;

FIG. 8a-8c show various views of another exemplary embodiment of a drug eluting member;

FIG. 9a-9c show various views of the drug eluting member in FIG. 8a;

FIG. 10 shows a perspective view of the drug eluting member in FIG. 8a attached to an intraocular lens;

FIG. 11 shows a perspective view of a device for holding any one of the drug eluting members shown in FIGS. 1 to 7;

FIG. 12a-12b show perspective views of the device in FIG. 11 used to receive a drug eluting member;

FIG. 13a-13d show perspective views of the steps to attach a drug eluting member using the device in FIG. 11;

FIG. 14 shows a flow diagram of a method of attaching a drug eluting member using the device in FIG. 11;

FIG. 15a-15c shows elevation views of the steps to fabricate a drug eluting member in any one of FIGS. 1 to 10;

FIG. 15-1a shows a sectional view of an exemplary embodiment of a mold for a drug eluting member in any one of FIGS. 1 to 10;

FIG. 15-1b shows a partial sectional view of a drug eluting member formed on the mold in FIG. 15-1a;

FIG. 15-2 shows a close up sectional view the drug eluting member in FIG. 15-1a;

FIG. 16 shows a flow diagram of a method of fabricating a drug eluting member in any one of FIGS. 1 to 10;

FIG. 17 shows a picture of an example of the matrix of the drug eluting member;

FIG. 18 shows a graph of a typical degradation profile of a drug eluting material;

FIG. 19 shows a graph of a typical mass loss profile of a drug eluting material; and

FIG. 20 shows a graph of a typical drug release profile from the intraocular lens.

### DETAILED DESCRIPTION

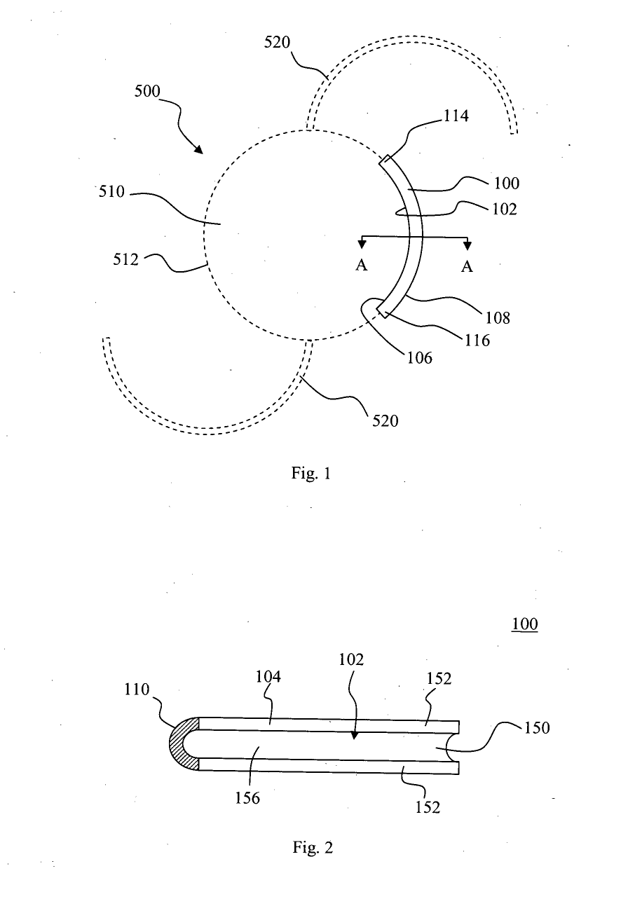

FIG. 1 shows an exemplary embodiment of a drug eluting member 100. Drug eluting member 100 is adapted to be attachable onto a perimeter edge 512 of a lens portion 510 of an intraocular lens 500 (shown in broken lines). Drug eluting member 100 includes an interfacing portion 102 which is adapted to receive a portion of the perimeter edge 512.

In other words, drug eluting member 100 may be attached onto the perimeter edge 512 of intraocular lens 500. Drug eluting member 100 has interfacing portion 102 which is able to receive a portion of the perimeter edge 512.

As shown in FIG. 1, the intraocular lens 500 has a lens portion 510 and a pair of haptics 520 extending from the lens portion 510 and curves radially towards the lens portion 510. Lens portion 510 may be known as an optic and it may be a transparent optical lens. Haptics 520 are generally flexible and enables the lens portion 510 to be centred to the axis or center of an eye when inserted into the eye. Lens portion 510 includes perimeter edge 512 surrounding the lens portion 510 thus defining the boundaries of the lens portion 512. Lens portion 510 may be divided into two half portions by an imaginary line through two points where the respective haptics 520 extend from the lens portion 510. Perimeter edge 512 may be circular such that the lens portion 510 is circular. Each of the two half portions may be semi-circular.

Drug eluting member 100 has an interfacing portion 102. Interfacing portion 102 may be a portion of the drug eluting member 100 that contacts or interfaces with the lens portion 510 when the drug eluting member 100 is attached to the lens portion 510 of the intraocular lens 500. Interfacing portion 102 may include a channel 150 (see FIG. 2) adapted to receive a portion of the perimeter edge 512 therein.

As shown in FIG. 1, drug eluting member 100 may have an inner rim 106 and an outer rim 108 opposite the inner rim 106. Drug eluting member 100 may include a first end 114 and a second end 116. Inner rim 106 and outer rim 108 may extend from the first end 114 to the second end 116. When the drug eluting member 100 is being attached to the lens portion 510, the inner rim 106 is the nearest edge to the centre of lens portion 510 while the outer rim 108 is further away from the centre of the lens portion than the inner rim 106. Outer rim 108 may be parallel to the inner rim 106.

FIG. 2 shows a sectional view of the drug eluting member 100 along line A-A in FIG. 1. As shown in FIG. 2, drug eluting member 100 may have a C-shaped cross section. Although a C-shaped cross section is shown, drug eluting member 100 may include other cross-sectional-shapes. Interfacing portion 102 of the drug eluting member 100 may include a facing side 104 at the inner rim 106. Facing side 104 is the side that faces the lens portion 510 when drug eluting member 100 is attached to the lens portion 510. Facing side 104 may be substantially perpendicular to the plane of the lens portion 510.

Drug eluting member 100 may include an outer surface 110. Outer surface 110 may be connected to the facing side 104. Surface 110 may extend from the facing side 104 to the outer rim 108. As outer surface 110 extends from the facing side 104 to the outer rim 108, surface 110 may be tapered towards the outer rim 108. In other words, outer surface 110 may form a semi-circular or the profile of an acute end of an elliptical, e.g. egg-shaped, cross section profile.

Channel 150 may be formed on the facing side 104 of the drug eluting member 100 such that facing side 104 is divided by the channel 150 thus forming a pair of lips 152 such that the channel is formed between the pair of lips 152. Channel 150 may include an inner surface 156 which connects to the facing side 104. Inner surface 156 may conform to and substantially parallel to the outer surface 110 such that the thickness of the drug eluding member 100 between the outer surface 110 and the inner surface 110 from one lip 152 to the other lip 152 may be substantially uniform.

Pair of lips 152 may be adapted to receive the lens portion 510 between them and within the channel 150. Lens portion 510 may be secured to the drug eluting member 100 by securing the lens portion 510 to the pair of lips 152 mechanically, e.g. by clamping the lens portion 510.

Interfacing portion 102 may include an adhesive surface 154 adapted to adhere the drug eluting member 100 to the lens portion 510. Adhesive surface 154 may be formed between the pair of lips 152. Adhesive surface 154 may be formed within channel 150 by coating the inner surface 156 with a layer of bio-adhesive.

Referring to FIG. 1, drug eluting member 100 may be arcuated between the first end 114 and the second end 116. As shown in FIG. 1 where the lens portion 510 has a circular profile, drug eluting member 100 may be arcuated to suit the curved profile of the portion of perimeter edge 512 of the lens portion 510. Accordingly, it can be seen that channel 150 of the drug eluting member 100 may conform to the profile of the portion of the perimeter edge 512 which the drug eluting member 100 is attached to. This would maximize the surface of adhesion between the drug eluting member 100 and the lens portion 510.

Drug eluting member 100 may be made from a bio-degradable material. As such, drug eluting member 100 may be bio-degradable.

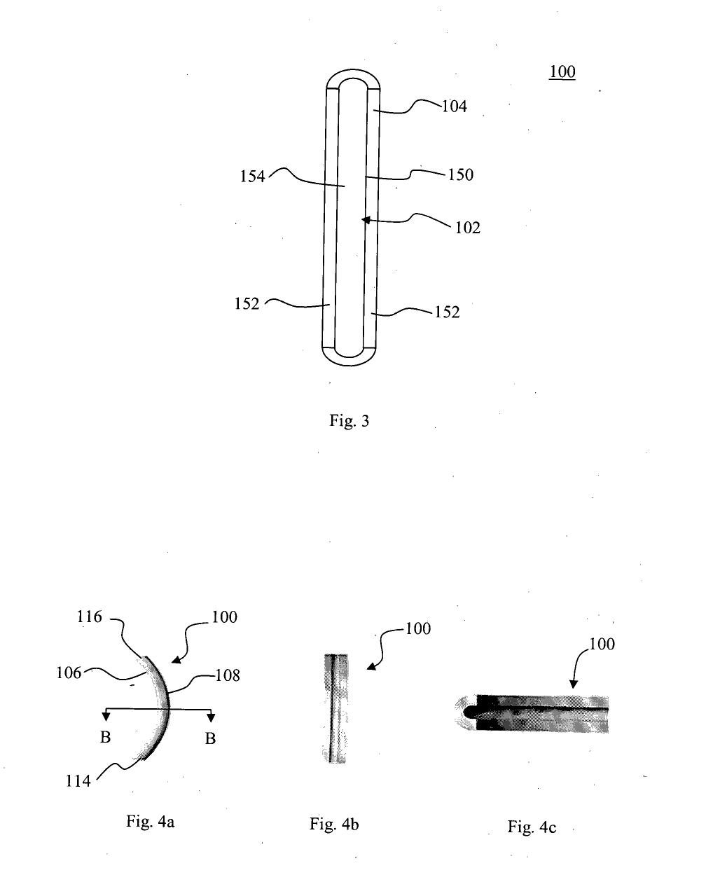

FIG. 3 shows a frontal view of the drug eluting member 100.

FIG. 4a-4c shows another exemplary embodiment of the drug eluting member 100. As shown in FIG. 4a, the first end 114 of the drug eluting member 100 may be tapered from the inner rim 106 to the outer rim 108 such that first end 114 has a side parallel to a side of the second end 116. As seen in FIG. 4a, first end 114 may be a pointed end. Similarly, second end 116 may be tapered from the inner rim 106 to the outer rim 108 to form a pointed end. FIG. 4c shows a cross section of the drug eluting member 100 in FIG. 4a along line B-B.

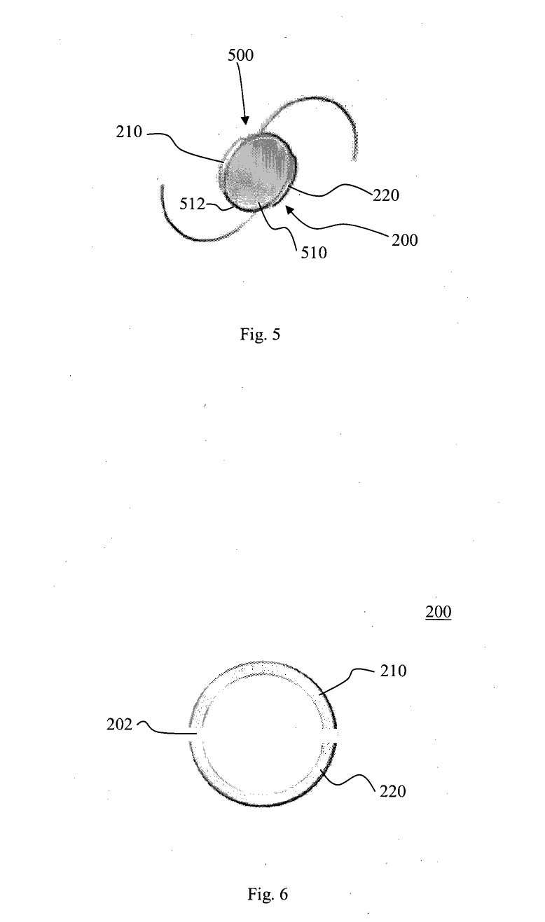

FIG. 5 shows the drug eluting member 100 in FIG. 4a being attached to the lens portion 510 of the intraocular lens 500. Drug eluting member 100 may be adhered to or clamped onto the perimeter edge 512 of the lens portion 510.

A drug eluting device 200 is shown in FIG. 5. Drug eluting device 200 may include a first drug eluting member 210 and a second drug eluting member 220. First drug eluting member 210 is adapted to be attached to a portion of a perimeter edge 512 of a lens portion 510 of an intraocular lens 500, and the second drug eluting member 220 being is adapted to be attached to another portion of the perimeter edge 512. As shown in FIG. 5, when drug eluting members 210,220 are being attached to the lens portion 510 of the intraocular lens 500, first drug eluting member 210 may be substantially opposite the second drug eluting member 220 and across the lens portion 510. This configuration is possible when there are two drug eluting members. First and second drug eluting members 210,220 may be attached to each of the two half portions of the lens portion 510 so that balance of lens portion 510 may be achieved when the intraocular lens 500 is inserted into the eye. However, it may be possible to have more than two drug eluting members 100 attached to the lens portion 510 and the drug eluting members may be spaced apart equally around the perimeter edge 512 of the lens portion 510.

FIG. 6 shows an exemplary embodiment of drug eluting device 200. As shown in FIG. 6, drug eluting device 200 may include two drug eluting member 210,220 which surrounds the lens portion 510 (not shown in FIG. 6) along the perimeter edge 512 of the lens portion 510. Drug eluting device 200 may substantially surround the lens portion 510 except leaving two gaps 202 for the extension of pair of haptics 520 (not shown in FIG. 6) from the lens portion 510. Drug eluting members 210,220, as mentioned above, may have channels (not shown in FIG. 5) for receiving the lens portion 510.

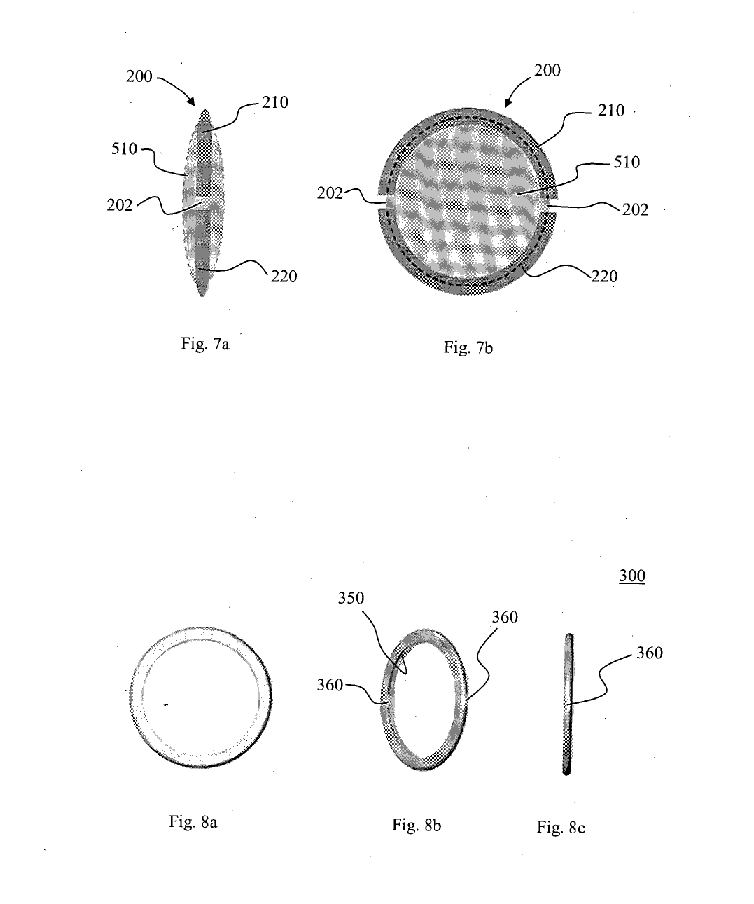

FIG. 7a-7b show various views of the embodiment of drug eluting device 200 in FIG. 6 being attached to the lens portion 510 of the intraocular lens 500. As shown, gaps 202 are formed between drug eluting members 210,220 for extension of the pair of haptics (not shown in FIG. 7a-7b) from the lens portion 510. Drug eluting members 210,220 may have any one of the same cross section profiles as described in earlier embodiments of drug eluting member 100.

FIG. 8a-8c show various views of another exemplary embodiment of drug eluting member 300. As shown in FIG. 8a, drug eluting member 300 may be ring-shaped. Referring to FIG. 8b, drug eluting member 300 may include at least one opening 360 for a haptic of the intraocular lens (not shown in FIG. 8b) to pass through when the drug eluting member 300 is attached to the lens portion 510. Drug eluting member 300 may include two opening 360. Two openings 360 may be located directly opposite each other along the drug eluting member 300. A skilled person would understand that there may be more than two openings 360 depending on the design of the intraocular lens 500, e.g. if there are more haptics or extrusions from the lens portion 510. Similar to the earlier embodiments, drug eluting member 300 may include a channel 350 for receiving the lens portion 510. Channel 350 may be formed along the facing side 504 of the ring-shaped drug eluting member 300. Drug eluting member 300 may have any one of the same cross section profiles as described in earlier embodiments of drug eluting member 100.

Drug eluting member 300 may be formed by two semi-circular drug eluting members, a first drug eluting member and a second drug eluting member, joined together to form the ring-shape. First drug eluting member and second drug eluting member may meet to form a through hole capable of surrounding the lens portion 510 of the intraocular lens 500 thereby attaching the first drug eluting member and second drug eluting member to the lens portion 510. It can be understood by a skilled person that the ring-shaped drug eluting member 300 may be formed by three or more drug eluting members. First drug eluting member and second drug eluting member may each include at least one opening 360 for a haptic of the intraocular lens (not shown in FIG. 8b) to pass through when the drug eluting member 300 is attached to the lens portion 510. Two openings 360 may be located directly opposite each other.

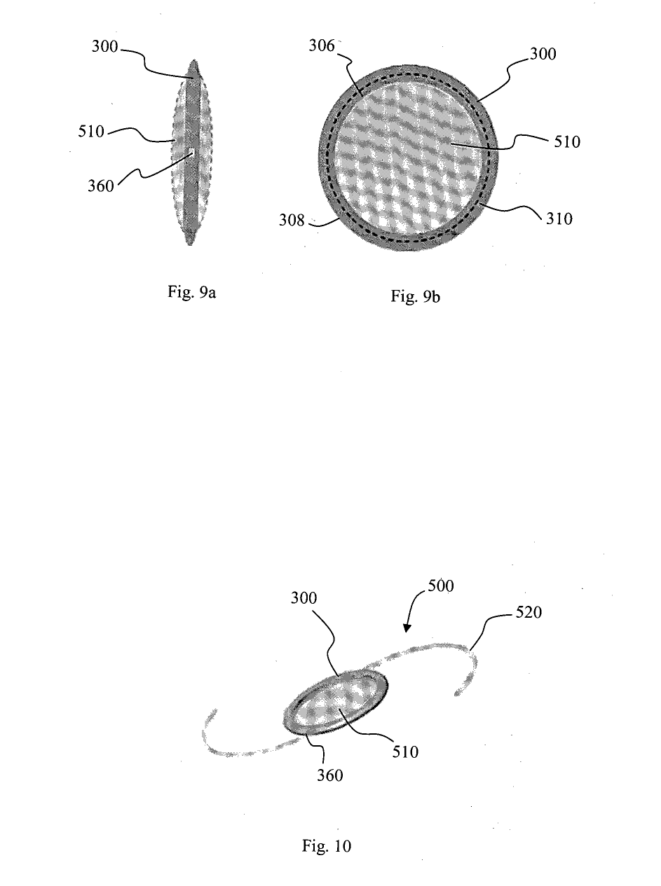

FIG. 9a-9b show a side view and front view of the drug eluting member 300 attached to the lens portion 510. As seen in FIG. 9a, drug eluting member 300 may have an outer surface 310 which conforms substantially to the profile of lens portion 510. Outer surface 310 may be tapered gradually from the inner rim 306 (see FIG. 9b) of the drug eluting member 300 to the outer rim 108 as described above.

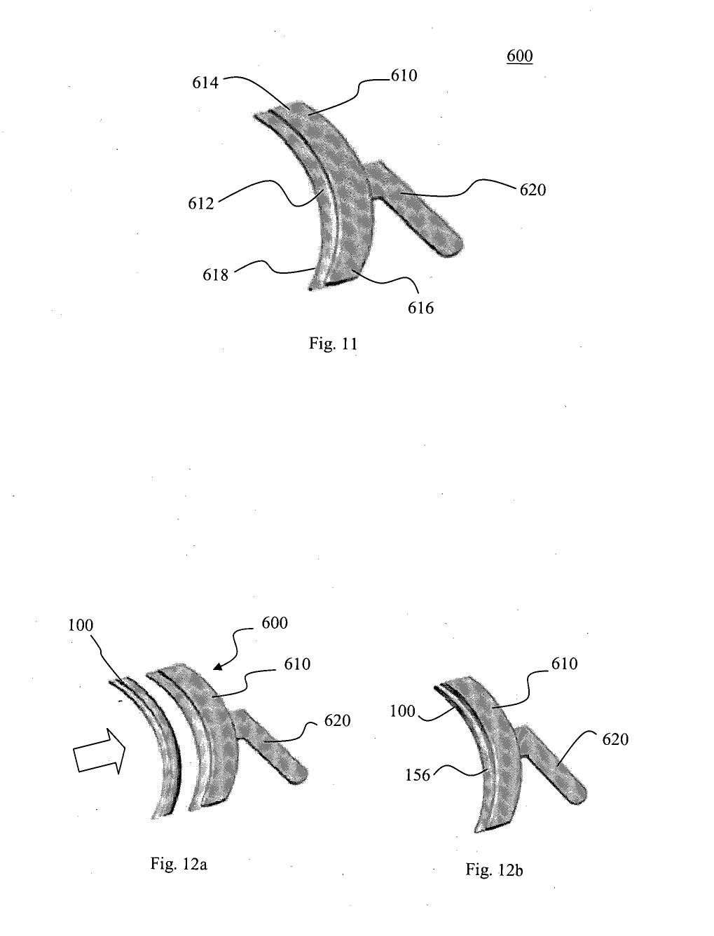

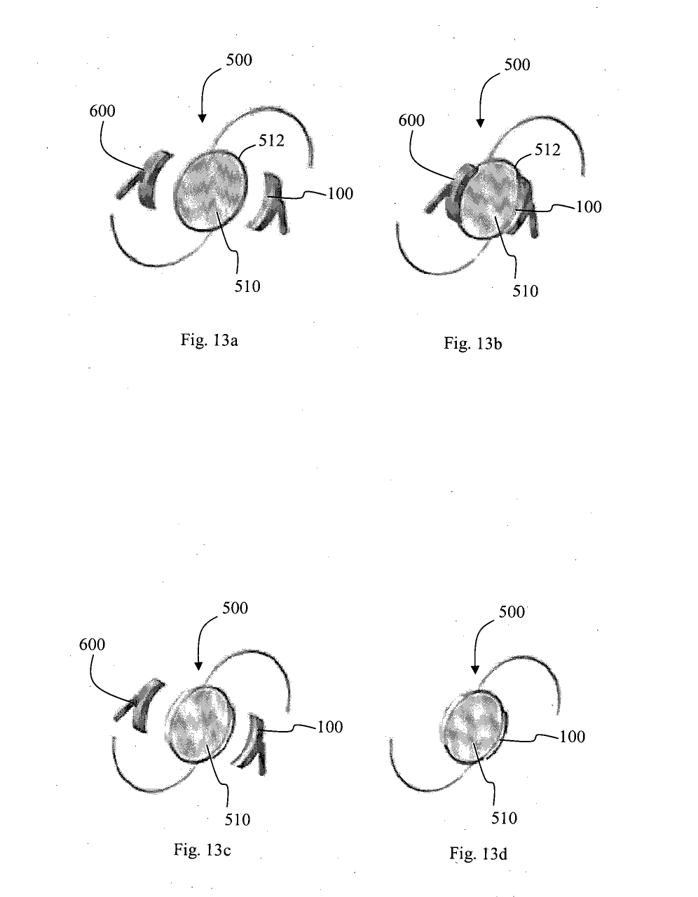

FIG. 10 shows drug eluting member 300 being attached to the intraocular lens 500. As for all drug eluting members described above, drug eluting member 300 may be made of an elastic material. Drug eluting member 300 may be fitted onto the intraocular lens 500 by bending the haptics 520 and/or stretching the drug eluting member 300. As clearly understood by a skilled person, it may not be necessary for drug eluting member 300 to have an adhesive layer as the ring-shaped drug eluting member 300 surrounds and straps onto the lens portion 510 when attached to it. As such, drug eluting member 300 may be attached to the lens portion 510 mechanically.

FIG. 11 shows a device 600 or drug eluting attaching device for holding any one of the embodiments of the drug eluting member described earlier. Device 600 may be used for holding a drug eluting member to a portion of the perimeter edge 512 of lens portion 510 of an intraocular lens 500. Device 600 may include a holder 610 which is adapted to hold the drug eluting member, e.g. drug eluting member 100, and a handling portion 620 for a user to handle the holder 610.

As shown in FIG. 11, holder 610 may have an arcuated profile. Holder 610 may include a receiving channel 612 for receiving the drug eluting member 100 therein. Receiving channel 612 may have an inner profile that conforms to the drug eluting member 100. Holder 610 may have a first end 614 and a second end 616. Holder 610 may arcuate from the first end 614 to the second end 616. Holder 610 may include a pair of lips 618 forming the receiving channel 612 between.

Handling portion 620 may be a portion or part of the holder 610. A user may hold the holder 610 at the handling portion 620 to attach drug eluting member 100 to the lens portion 510. Handling portion 620 may include a handle extending from the holder 610 as shown in FIG. 11.

FIG. 12a-12b show device 600 receiving arcuated drug eluting member 100. Drug eluting member 100 may be ring-shaped drug eluting member 300.

Drug eluting member 100 may be attached onto the perimeter edge 512 of the lens portion 510 of the intraocular lens 500 (not shown in FIG. 12a-12b) as two halves e.g. drug eluting device 200. Device 600 allows the drug eluting member 100 to be attached onto the perimeter edge 512 of lens portion 510 before loading the intraocular lens 500 into an intraocular lens injector (not shown in FIG. 12a-12b) and injecting the intraocular lens 500 into the capsular bag in an eye.

A thin layer of bio-adhesive may be coated onto the inner surface 156 of the drug eluting member 100 for securing the drug eluting member 100 onto the lens portion 510.

Drug eluting member may be made from bio-stable polymers or similar polymer. Bio-stable polymer may include poly 2-phenethyl methacrylate (poly(PhEMA)), poly ethyl-methacrylate (PEMA), poly 2,2,2-trifluoroethyl methacrylate (PTFEMA), poly dimethylsiloxane (PDMS), poly diphenylsiloxane (PDPhS), poly ethylene vinyl acetate (PEVA), polyurethanes or poly ethylene terephthalate.

Drug eluting member may be made from biodegradable polymers e.g. a poly(a-hydroxy ester) or a biodegradable polyurethane which contains a PLA/PCL copolymer or a PCL/PTMC copolymer as the soft blocks. Biodegradable polymers may have substantial mass loss starting at around 1 month to 6 months, and being substantially absorbed within 3-12 months. The base material used for the drug eluting member may be bio-degradable elastomer.

FIG. 13a-13d shows a method 1000 of attaching a drug eluting device 200 to the lens portion 510 of an intraocular lens 500 using device 600. Drug eluting device 200 may include two drug eluting members 100 as shown in FIG. 13a. Drug eluting device 200 may include more than two drug eluting members 100.

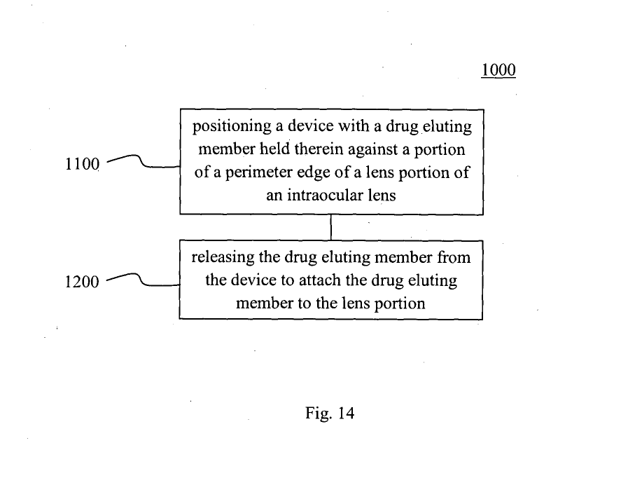

FIG. 14 shows a flow diagram of method 1000. Method 1000 includes positioning the device 600 with a first drug eluting member 210 held therein against a portion of the perimeter edge 512 of the lens portion 500 as shown in step 1100. Drug eluting member 100 is released from the device 600 to attach the drug eluting member 100 to the lens portion 510 in step 1200.

Two devices 600 may be used hold the two drug eluting members 100 of drug eluting device 200. As shown in FIG. 13a, devices 600 may be held adjacent the intraocular lens 500 in preparation of attaching the drug eluting members 100. Referring to FIG. 13b, each device 600 may be positioned, with the drug eluting member 100 held within the device 600, against a portion of the perimeter edge 512. Once the drug eluting members 100 are secured onto the respective portions of the perimeter edge 512 of the lens portion 510, the drug eluting members 100 may be released from the device 600 by pulling the device 600 away from the lens portion 510 as shown in FIG. 13c. As the drug eluting devices 100 are being adhered to the lens portion 510, the adhering force between the drug eluting members 100 and the lens portion 510 should be higher than the holding force between the devices 600 and the drug eluting members 100. Drug eluting member 100 may also be secured to the lens portion 510 by clamping. FIG. 13d shows the drug eluting members 100 being attached to the lens portion 510.

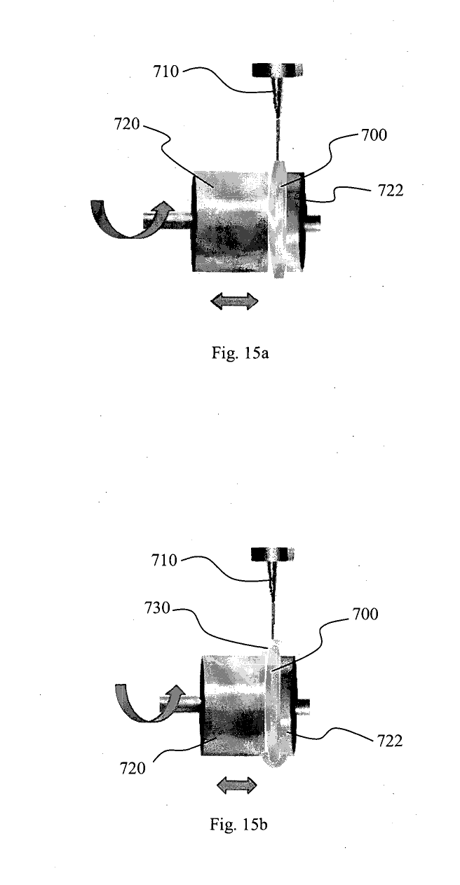

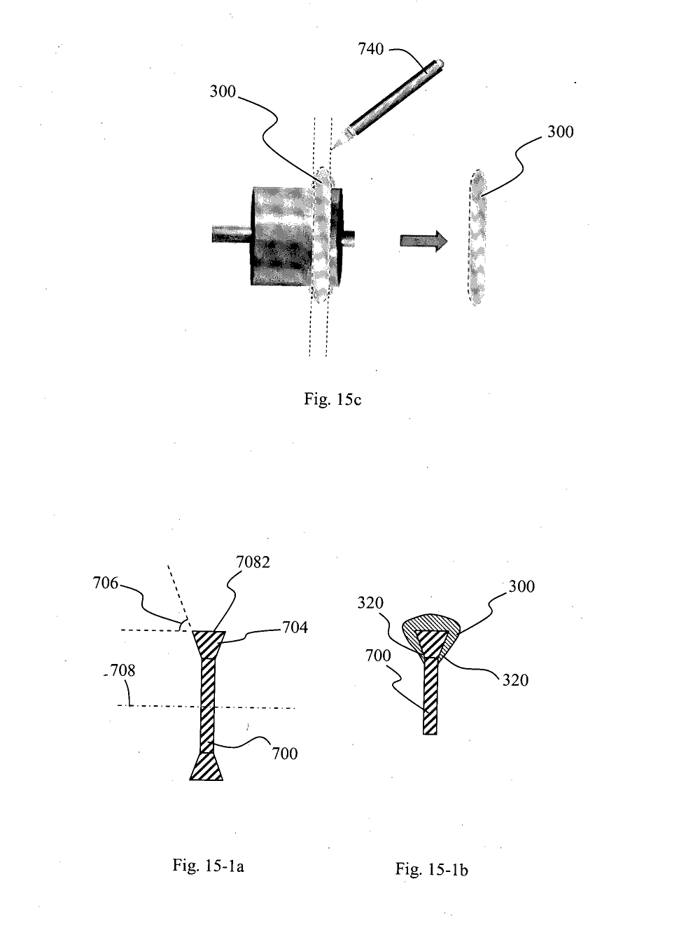

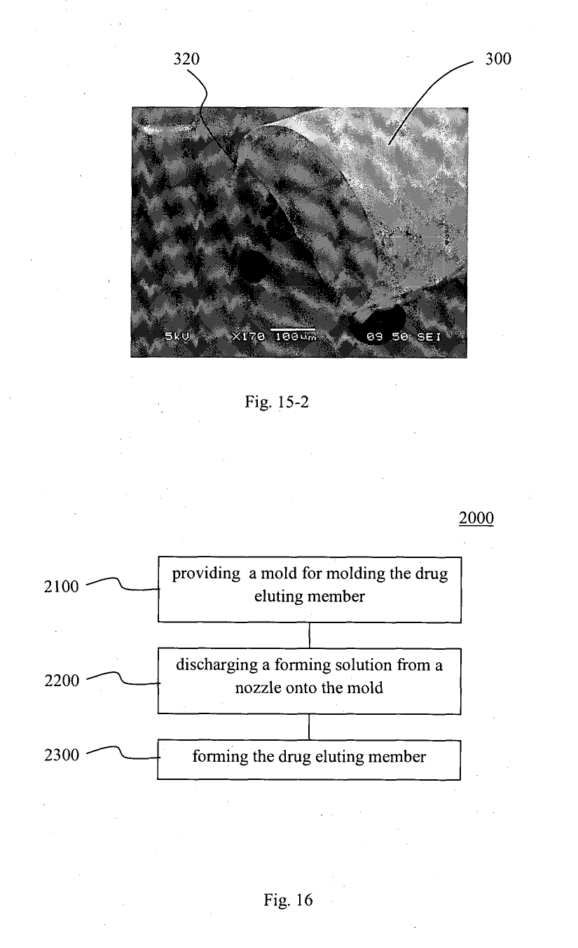

FIG. 15a-15c show a method 2000 of fabricating a drug eluting member as mentioned in any one of the embodiments above, e.g. drug eluting member 300 having a ring-shaped profile.

FIG. 16 shows a flow diagram of method 2000. As shown in FIG. 16 (and referring to FIG. 15a-15c), method 2000 includes providing a mold 700 for molding the drug eluting member 300 in step 2100. In step 2200, a forming solution 730 is discharged from a nozzle 710 onto the mold 700. In step 2300, the drug eluting member 300 is formed on the mold 700.

Referring to FIG. 15a, mold 700 may be mounted in between two mandrels 720,722 which may then be fixed onto a spray coating machine (not shown in FIG. 15a-15c). Mold 700 may be of the same dimension as the intraocular lens 500. Mold 700 may be a disc-shaped plate, e.g. a flat plate with parallel circular sides or a disc with an augmented perimeter portion (as described below).

Mandrel 730 may be set to move in both rotational and translational motion. (see arrows in FIG. 15a). A forming solution 730 may be discharged from the nozzle 710 onto mold 700.

Forming solution 730 may include a dissolved/dispersed polymer and a drug solution. Forming solution 730 may be sprayed from nozzle 710 onto mold 700. Forming solution 730, or drug dispersed polymer solution, would dry upon being coated onto the mold 700.

As shown in FIG. 15b, forming the drug eluting member 300 may include displacing the mold 700 with respect to the nozzle 710 and coating the mold 700 with the forming solution 730.

Displacing the mold 700 may include rotating the mold 700 at about an axis and/or translating the mold 700 along the axis. A number of cycles of rotational and/or translational movement may be pre-determined to fabricate drug eluting member 300 with a desired thickness of the coating, i.e. desired thickness of drug eluting member 100.

Drug eluting member 300 may be dried. Drug eluting member may be dried in a vacuum oven (not shown in FIG. 15) at about 37° C. over a period of about 7 days or more.

Referring to FIG. 15c, drug eluting member 300 may then be shaped. The excess coated polymer may then be trimmed off by a shaping tool 740, e.g. a sharp tool like a knife. The finished drug eluting member 300 may be easily removed from the mold 700 due to the elastic property of the drug eluting member 300. As such, drug eluting member 300 may be made from an elastic material.

Mold 700 may have augmented perimeter portion 704. FIG. 15-1a shows a sectional view of mold 700 with augmented perimeter portion 704. Sectional view of mold 700 may resemble a “dumbbell”. From the sectional view, it can be seen that circular surface of mold 700 tapers outwardly away from centre plane of the mold 700 and from the centre of the mold 700 towards the perimeter of mold 700 such that the thickness at the edge of mold 700, i.e. at the augmented perimeter portion, may be larger than the thickness of the mold 700 at the centre portion of the mold 700. As such, mold 700 may have a thicker perimeter portion than the centre portion of the mold 700. As shown in FIG. 15-1a, an edge 7082 parallel to a centre axis 708 at the perimeter forms an angle 706 with a tapered surface portion 7084 of the augmented perimeter portion 704. Angle 706 may be from 45° to 80°.

FIG. 15-1b shows a partial sectional view of a drug eluting element 300 formed on mold 700 with augmented perimeter portion 704. As shown, drug eluting element 300 that is formed on mold 700 with augmented perimeter portion 704 may have a pair of jaws 320 which extends inwards towards each other so that drug eluting member 300 may provide better hooking or attaching property to the lens portion 510 of intraocular lens 500. FIG. 15-2 shows a perspective view of drug eluting element 300 molded by a mold with augmented perimeter portion 704.

Drug eluting member 300 may be evaluated in vitro for drug release and degradation. A set of the drug eluting member 300 may then be attached, e.g. adhered, to the intraocular lens 500 and sterilized using ethylene oxide. The drug eluting member 300 may be able to bend and be implant together with the intraocular lens 500 by an intraocular lens injector, without being detached from the intraocular lens 500.

Drug eluting member 300 may be fabricated by the spray coating technique due to the delicate nature and stringent dimension of the drug eluting member 300. Drug eluting member 300 may be fabricated by dip coating techniques, or ultra-sonic spray coating technique. Such a method allows dimension of drug eluting member to be in micron scale.

As shown above, drug eluting member may be attached mechanically or using a bio-adhesive to the edge of an intraocular lens. A skilled person would appreciate that drug eluting member would not affect the optical properties of the intraocular lens or distort the lens orientation post-operatively.

Drug eluting member may be formulated to have drugs dispersed or dissolved throughout the polymer (“matrix” system). Drugs commonly used to treat postoperative infection include: (1) Levofloxacin; (2) Moxifloxacin; and (3) Gatifloxacin.

Levofloxacin is a synthetic antibiotic of the fluoroquinolone drug and is used to treat bacterial eye infections by oral or topical administration. It works to treat bacterial infections by interfering with an enzyme that the bacteria need to multiply.

Moxifloxacin and gatifloxacin belongs to the fourth generation of the fluoroquinolones, which are commonly used in the United States. Clinically, these two drugs showed better ocular tissue penetration and improved spectrum of bacteria coverage. It also appears that moxifloxacin is superior to gatifloxacin in terms of penetration.

TABLE 1
Chemical structures of levofloxacin, moxifloxacin and gatifloxacin.
Levofloxacin

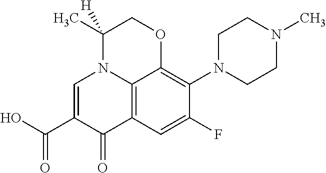 [SMILES: C[C@H]1COc2c(N3CCN(C)CC3)c(F)cc3c(=O)c(C(=O)O)cn1c23]

Moxifloxacin

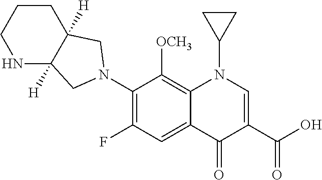 [SMILES: COc1c(N2C[C@@H]3CCCN[C@@H]3C2)c(F)cc2c(=O)c(C(=O)O)cn(C3CC3)c12]

Gatifloxacin

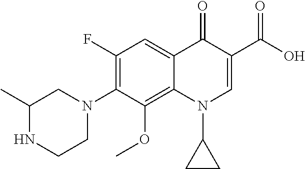 [SMILES: COc1c(N2CCNC(C)C2)c(F)cc2c(=O)c(C(=O)O)cn(C3CC3)c12]

Approximately 0-50% of a drug, such as levofloxacin, moxifloxacin and gatifloxacin may be dispersed or dissolved in the polymer matrix body.

Drugs may include anti-inflammatories, anti-cancer drugs, as well as antibiotics with both hydrophobic and hydrophilic properties.

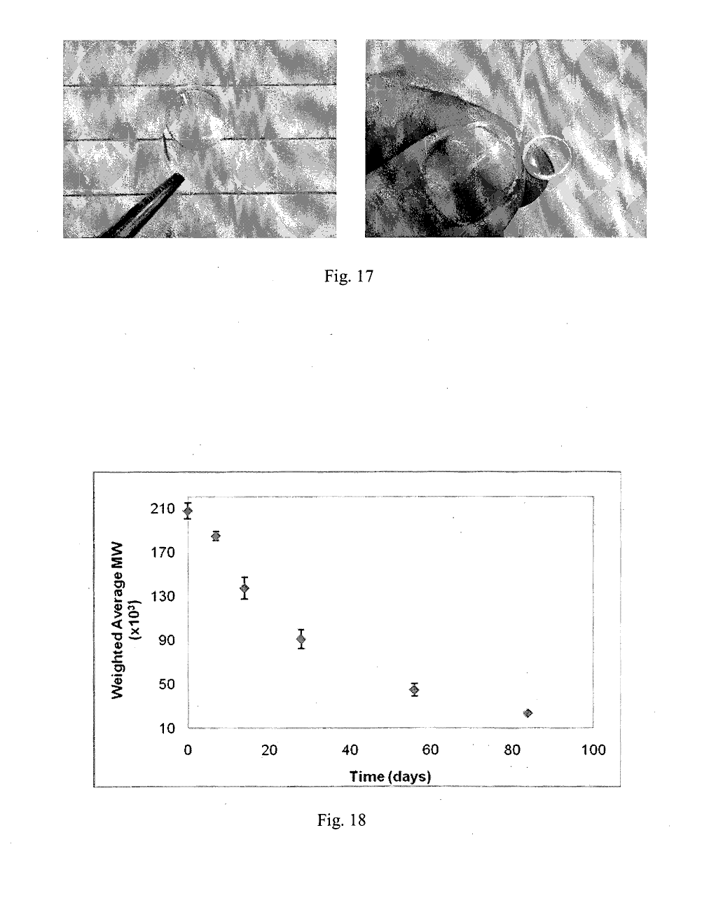

When the drugs are formulated into drug eluting member, a loading of 5% to 50% by weight may be proposed. Such drugs are released in a controlled fashion from the “matrix”, as shown in FIG. 17.

FIG. 18 shows a typical degradation profile of a drug eluting material.

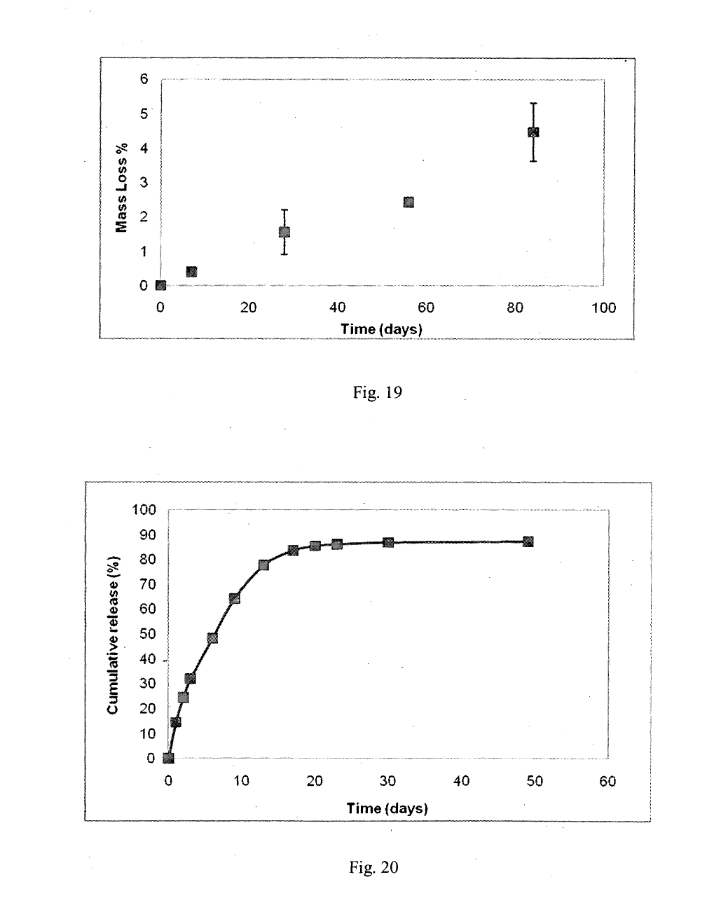

FIG. 19 shows a typical mass loss profile of a drug eluting material.

FIG. 20 shows a typical drug release profile from the intraocular lens.

A typical composition for a fully-degradable drug eluting member with drugs surrounds the edge of the IOL would be as follows:
Poly lactide co caprolactone, with LA/CL ratio of (70:30), (60:40) and (50:50).
Poly lactide co TMC-caprolactone co poly lactide tri-block polyurethane, with LA/TMC/CL ratio of (0.38:1:1), (0.75:1:1), (1.5:1:1), (1.5:0.33:1) and (1:0.33:1).
15% to 30% of levofloxacin by weight.
15% to 30% of moxifloxacin by weight.
15% to 30% of gatifloxacin by weight.
Block copolymer poly ethylene glycol co lactide, with PEG length of 100-10,000 Da, and lactide length of 500-15,000 Da.
Block copolymer poly ethylene glycol co caprolactone, with PEG length of 100-10,000 Da and caprolactone length of 500-15,000 Da.

1. A drug eluting member adapted to be attachable onto a perimeter edge of a lens portion of an intraocular lens, the drug eluting member comprising:
an interfacing portion adapted to receive a portion of the perimeter edge.

2. The drug eluting member of claim 1, wherein the interfacing portion comprises a channel adapted to receive the portion of perimeter edge therein.

3. The drug eluting member of claim 2, wherein the channel conforms to the profile of the portion of the perimeter edge.

4. The drug eluting member of claim 1, wherein the interfacing portion comprises an adhesive surface adapted to adhere the drug eluting member to the lens portion.

5. The drug eluting member of claim 1, wherein the drug eluting member is bio-degradable.

6. The drug eluting member of claim 1, wherein the drug eluting member is arcuated.

7. The drug eluting member of claim 1, wherein the drug eluting member surrounds the lens portion along the perimeter edge of the lens portion.

8. The drug eluting member of claim 1, wherein the drug eluting member is ring-shaped.

9. A drug eluting device comprising a first drug eluting member and a second drug eluting member, the first drug eluting member being adapted to be attached to a first portion of a perimeter edge of a lens portion of an intraocular lens, and the second drug eluting member being adapted to be attached to a second portion of the perimeter edge, wherein the first drug eluting member comprises a first interfacing portion adapted to receive said first portion of the perimeter edge, and the second drug eluting member comprises a second interfacing portion adapted to receive said second portion of the perimeter edge.

10. The drug eluting device of claim 9, wherein, when attached to the lens portion, the first drug eluting member is substantially opposite the second drug eluting member.

11. The drug eluting member of claim 9, wherein the first drug eluting member and second drug eluting member meet to form a through hole capable of surrounding the lens portion of the intraocular lens thereby attaching the first eluting member and second eluting member to the lens portion.

12. A device for holding a drug eluting member to a portion of a perimeter edge of a lens portion of an intraocular lens, the drug eluting member adapted to be attachable onto the perimeter edge of the lens portion of the intraocular lens, and the drug eluting member comprises an interfacing portion adapted to receive said portion of the perimeter edge, the device comprising:
a holder adapted to hold the drug eluting member; and
a handling portion for a user to handle the holder.

13. The device of claim 12, wherein the holder comprises a receiving channel for receiving the drug eluting member therein.

14. The device of claim 12, wherein the handling portion includes a handle extending from the holder.

15. A method of attaching a drug eluting member to a perimeter edge of a lens portion of an intraocular lens using a device for holding the drug eluting member to a portion of the perimeter edge of the lens portion of the intraocular lens, the device comprising a holder adapted to hold the drug eluting member and a handling portion for a user to handle the holder, and the drug eluting member comprises an interfacing portion adapted to receive said portion of the perimeter edge, the method comprising:
positioning the device with the drug eluting member held therein against a portion of the perimeter edge; and
releasing the drug eluting member from the device to attach the drug eluting member to the lens portion.

16. The method as claimed in claim 15, further comprising adhering the drug eluting member to the lens portion.

17. A method of fabricating a drug eluting member adapted to be attachable onto a perimeter edge of a lens portion of an intraocular lens, the drug eluting member comprising an interfacing portion adapted to receive a portion of the perimeter edge, the method comprising:
providing a mold for molding the drug eluting member;
discharging a forming solution from a nozzle onto the mold; and
forming the drug eluting member.

18. The method of claim 17, wherein forming the drug eluting member comprises,
displacing the mold with respect to the nozzle; and
coating the mold with the forming solution.

19. The method of claim 18, wherein displacing the mold comprises,
rotating the mold at about an axis; and/or
translating the mold along the axis.

20. The method of claim 17, further comprising shaping the drug eluting member.

21. The method of claim 17, wherein the mold is a disc-shaped plate.

22. The method of claim 17, wherein the mold is a disc-shaped plate with an augmented perimeter portion, wherein the thickness of the mold at the augmented perimeter portion is larger than the thickness of the mold at the centre portion.

23. The method of claim 17, wherein the forming solution comprises a polymer and drug solution.
